(19) United States
(12) Patent Application Publication
(10) Pub. No.: US 2008/0023010 A1
(43) Pub. Date: Jan. 31, 2008

(54) RF Shielding In MRI For Safety Of Implantable Medical Devices

(75) Inventors: D. Michael Inman, Gainsville, FL (US); Steven E. Maschino, Seabrook, TX (US)

Correspondence Address: CYBERONICS, INC., LEGAL DEPARTMENT, 6TH FLOOR, 100 CYBERONICS BOULEVARD, HOUSTON, TX, 77058, US

(73) Assignee: CYBERONICS, INC., Houston, TX (US)

(21) Appl. No.: 11/460,266

(22) Filed: Jul. 27, 2006

(51) Int. Cl. A61F 5/37

(52) U.S. Cl. 128/846

(57) ABSTRACT

Methods and apparatus for reducing the heating of an implantable medical device due to RF energy. An RF shield is disclosed which provides localized RF shielding of an implantable medical device while allowing other portions of a patient's body to be exposed. The RF shield described is made from an RF energy absorbing fabric which circumferentially wraps around a portion of a patient's body. The RF energy absorbing fabric can be composed of carbon fibers, conductive metal fibers, or combinations thereof. An advantage of the disclosed RF shield is that it need not be implanted within a patient.

### CROSS-REFERENCE TO RELATED APPLICATIONS

Not applicable.

### BACKGROUND

1. Technical Field

The subject matter of this disclosure generally relates to the field of implantable medical devices. More specifically, the present disclosure relates to reducing heat generated in an implantable medical device during imaging such as magnetic resonance imaging.

2. Background Information

Magnetic resonance (MR) imaging (MRI) uses radiofrequency (RF) waves and a strong magnetic field rather than x-rays to provide remarkably clear and detailed pictures of internal organs and tissues. The technique has proven very valuable for the diagnosis of a broad range of pathologic conditions in all parts of the body including cancer, heart and vascular disease, stroke, and joint and musculoskeletal disorders. MRI requires specialized equipment and expertise and allows evaluation of some body structures that may not be visible in similar detail with other imaging methods.

Certain implantable medical devices (IMDs) contain conductive elements that may heat up upon being exposed to RF energy from an MRI machine. One such conductive element is the helical-shaped conductor coil (i.e., lead). This component conducts current from the battery powered IMD to the tissue-stimulating electrode portion of the device. During an MRI scan, an RF-induced current can develop in the helical conductor coil and this can cause heating of tissue at the electrode portion of the IMD. Many MRI scans are performed on an area of the body remote from the IMD, yet due to the design of the MRI system, high levels of RF energy are still directed to the implant and may cause the device and the surrounding tissue to warm up.

Many RF shielding systems consist of a conductive box forming a “faraday cage” around the volume to be shielded. However, shielding the entire body would preclude effective imaging using the MRI scanner. Highly conductive shields both absorb and reflect radio energy. In the case of an open box shield, reflection is not desired, since it may actually serve to focus energy on the IMD. Thus, there is a need for an RF shield that reduces the heating of an IMD caused by RF energy, yet at the same time allows unfettered MRI imaging of unshielded portions of the body.

### BRIEF SUMMARY

The present disclosure addresses the issues noted above by providing an RF shield which can provide localized shielding of an IMD while allowing other portions of a patient's body to be exposed. The RF shield described herein is made from an RF energy absorbing fabric which wraps around a portion of a patient's body. One of the advantages of the disclosed RF shield is that it need not be implanted within a patient.

In at least one embodiment, an RF shield comprises a fabric comprising a plurality of carbon fibers. The fabric circumferentially surrounds a portion of a patient and reduces heating of an implantable medical device inside said patient due to RF energy.

In another embodiment, an RF shield comprises a fabric comprising a plurality of conductive metal fibers. The fabric circumferentially surrounds a portion of a patient's body and reduces heating of an implantable medical device inside the patient's body due to RF energy.

In another embodiment, a method comprises providing an RF shield made of an RF energy absorbing fabric. The method also comprises circumferentially surrounding at least a portion of a patient with said RF shield so as to reduce heating of an implantable medical device inside said patient.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a detailed description of the preferred embodiments of the invention, reference will now be made to the accompanying drawings in which:

FIG. 1 illustrates an embodiment of an RF shield;

FIG. 2 illustrates an implantable medical device that may be used with the RF shield;

FIG. 3 illustrates another embodiment of an RF shield;

FIG. 4 is a close up of the carbon fiber fabric suitable for use in an RF shield;

FIGS. 5A, 5B, and 5C illustrate different types of weaves that may be incorporated in an RF shield; and

FIG. 6 illustrates an embodiment of an RF shield including a first and second layer.

### DETAILED DESCRIPTION OF THE PREFERRED EMBODIMENTS

Certain terms are used throughout the following description and claims to refer to particular system components. This document does not intend to distinguish between components that differ in name but not function. In the following discussion and in the claims, the terms “including” and “comprising” are used in an open-ended fashion, and thus should be interpreted to mean “including, but not limited to . . . ”.

The present invention is susceptible to implementation in various embodiments. The disclosure of specific embodiments, including preferred embodiments, is not intended to limit the scope of the invention as claimed unless expressly specified. In addition, persons skilled in the art will understand that the invention has broad application. Accordingly, the discussion of particular embodiments is meant only to be exemplary, and does not imply that the scope of the disclosure, including the claims, is limited to specifically disclosed embodiments.

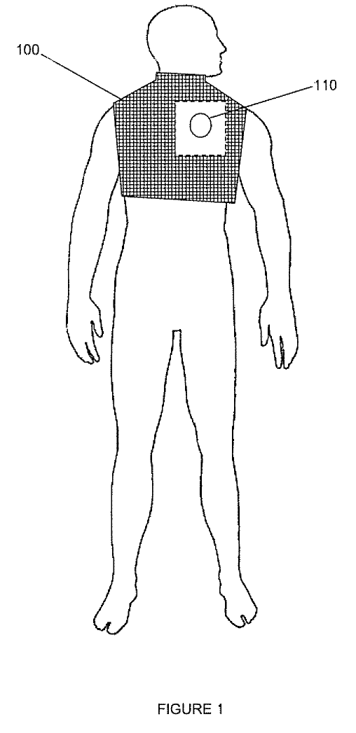

FIG. 1 illustrates an embodiment of an RF shield 100. Generally, embodiments of an RF shield 100 comprise a fabric that surrounds a portion of a patient's body in which an implantable medical device 110 is implanted. The RF shield 100 provides localized protection for the portions of the body which expose the implantable medical device to RF energy while leaving other parts of the patient's body exposed for MRI imaging. For example, an implantable medical device 110 typically is located in the upper torso of a patient. FIG. 1 depicts IMD 110 in cutaway view solely to show the presence of the IMD in the patient's body. It should be appreciated that IMD 110 is actually surrounded by RF shield 100. Thus, the embodiment of RF shield 100 shown in FIG. 1 is configured to circumferentially surround the patient's upper torso (including IMD 110), but leave the arms exposed. The shield described herein provides protection from RF energy originating from an MRI machine or any other source of RF energy.

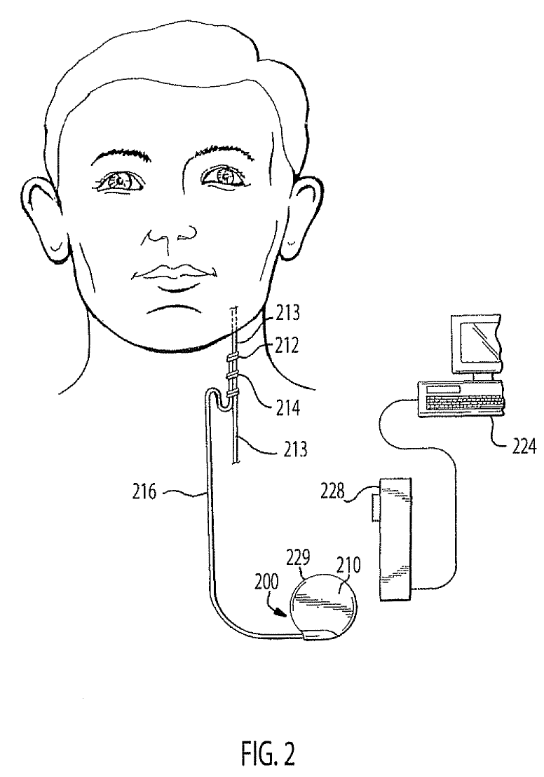

Generally, the term “implantable medical device” refers to any artificial device placed inside the human body, usually surgically. In a specific embodiment, the IMD may comprise a vagus nerve stimulator (VNS) system. FIG. 2 schematically illustrates an IMD 110 comprising a VNS system implanted in a patient. The vagus nerve stimulation system is representative of any of a variety of medical devices that may be subject to RF-induced heating during an MRI procedure. In at least one preferred embodiment, the IMD 110 comprises a vagus nerve signal generator 210 for applying an electrical signal to a vagus nerve 213, although electrical signals may be applied to other cranial nerves (e.g., the trigeminal and/or glossopharyngeal nerves) in other IMD systems. In the vagus nerve stimulation system of FIG. 2, lead assembly 216 is coupled to the signal generator 210 at a proximal end of the lead, and includes one or more electrodes, such as electrodes 212 and 214, at a distal end thereof. A conductive outer shell 229 of signal generator 210 may also be used as an electrode. The electrodes 212, 214 and 229 are used to stimulate (i.e., apply an electrical signal to) and/or sense the electrical activity of the associated tissue (e.g., the vagus nerve 213). The IMD also typically is capable of transcutaneously communicating with an external programming device 224 via a wand 228. Via the wand 228, the programming device 224 generally monitors the patient and the performance of the IMD 110 such as signal generator 210, and downloads new programming information into the device to alter its operation as desired. Other examples of appropriate medical devices which may be shielded by RF shield 100 include, without limitation, pacemakers, artificial hearts, defibrillators, ventricular assist devices, and the like.

In the embodiment shown in FIG. 1, fabric further forms a sleeveless garment which substantially covers the upper torso and the neck of the patient, but leaves the arms exposed. RF shield 100 is generally continuous about the portion of patient's body containing the implantable medical device since the MRI machine directs RF energy completely around the body. In one embodiment, RF shield 100 is configured much like a sweater or a turtleneck shirt in which the patient slips the garment over his or her head. Generally, RF shield 100 is conforms to the patient's body, but is preferably not so fitted as to constrict blood flow or the patient's breathing.

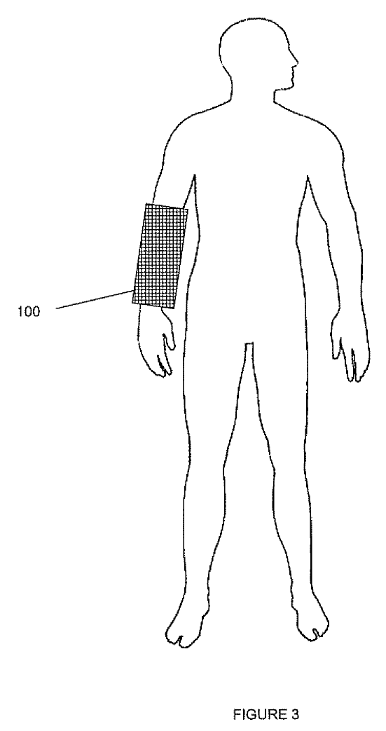

In another embodiment, RF shield 100 is configured like a vest or lifejacket (not shown). In such an embodiment, the patient inserts his or her arms through armholes in the garment. RF shield 100 is then fastened together at the front of the patient to form a continuous shield around upper torso and neck of the patient. RF shield 100 includes any type of fasteners including without limitation, zippers, clasps, Velcro, hooks, clips and the like. The fasteners are preferably made of polymeric materials so as not to absorb or reflect RF energy. In another embodiment, RF shield 100 is a sleeve which covers some or all of an appendage such as an arm or leg as shown in FIG. 3.

In preferred embodiments, the fabric is capable of absorbing and/or dissipating RF energy. In at least one embodiment, the fabric is also partially electrically conductive and non-magnetic. Partially conductive materials can absorb RF energy with minimal or no reflection. In the case of an MRI environment, the frequency of RF energy is known, and the fabric shield can be specifically tuned or constructed to absorb, but not reflect the specific wavelength. The RF energy absorbed by the fabric heats the garment instead of heating the implant or the patient's body. The absorption of the shield can be specifically tuned to the RF frequency of the MRI by manipulating the length and orientation of the fibers in the RF energy absorbing fabric. In at least one preferred embodiment, the fabric is capable of absorbing RF energy in the range of about 1 MHz to about 1 GHz, more preferably in the range of about 10 MHz to about 100 MHz. Complete (i.e., 100%) absorption of the RF energy is not necessary for the shield to perform its function. Even a minor absorption of RF energy by the RF shield reduces the production of heat in an implantable medical device. In an embodiment, the RF shield absorbs at least about 50% of the RF energy, more preferably at least about 70% of the RF energy.

In a preferred embodiment, the fabric comprises carbon fiber. The carbon fiber is preferably woven to form a mesh. In some embodiments, the fabric additionally comprises a plurality of conductive metal fibers. Examples of suitable conductive metals include without limitation, aluminum, gold, silver, copper or the like. Alternatively, the conductive metal fibers are coated with a resin.

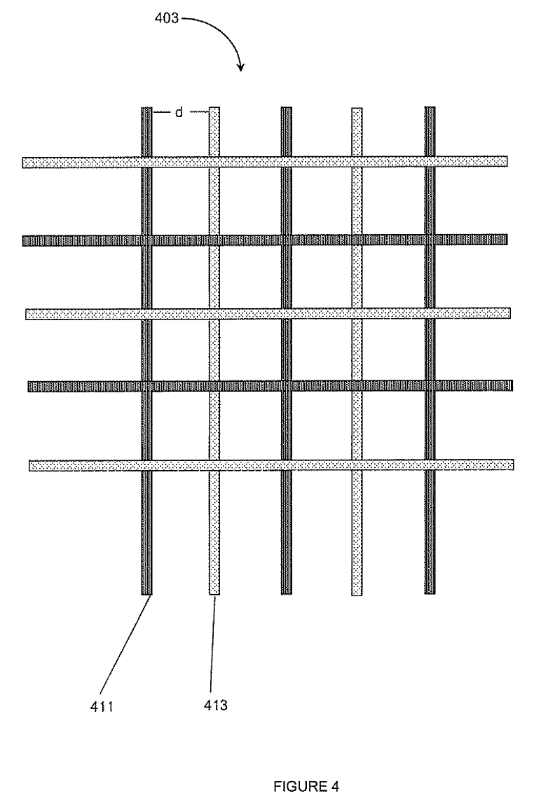

FIG. 4 is a close-up of an embodiment of a RF energy absorbing fiber. The distance, d, between each fiber (i.e., the diameter of the apertures in a mesh) is preferably in a range of from about 0.001 mm to about 2.0 cm, more preferably from about 0.01 mm to about 5 mm (see FIG. 4). In general, the smaller the distance between the fibers, the greater the density of carbon fiber in the fabric and therefore, the greater the RF absorption of the fabric. The diameter of each carbon fiber is preferably in the range of about 0.0001 mm to about 1.0 mm, more preferably in the range of about 0.005 mm to about 0.5 mm. Additionally, different embodiments of the RF shield may have carbon fiber fabrics of different thicknesses due to the weaving of the carbon fibers. The thickness of the composite carbon fiber fabric preferably ranges from about 1 mm to about 3 mm.

In one embodiment, the fabric is comprised entirely of carbon fibers. In another embodiment, the fabric comprises a plurality of conductive metal fibers 411 interwoven with a plurality of carbon fibers 413, as shown in FIG. 4. The conductive metal fibers 411 are preferably interwoven such that the fabric comprises alternating carbon fibers 413 and conductive metal fibers 411. In such an embodiment, the fabric comprises about 50% conductive metal, more preferably 75% conductive metal. In alternative embodiments, more carbon fibers 413 than conductive metal fibers 411 are employed, or vice versa, in a defined ratio, e.g. twice as many carbon fibers as conductive metal fibers 411. In addition, the fabric may comprise elastic fibers interwoven with the carbon fiber and/or metal fiber to impart elasticity and added flexibility to the fabric. The interwoven elastic fibers may provide added comfort to the patient as well as assist the fabric in conforming to the patient's body.

In alternative embodiments, the carbon filament is coated with a conductive metal, or metal alloy. A more detailed description of such coated filaments is found in U.S. Pat. No. 5,827,997, entitled “Metal Filaments for Electromagnetic Interference Shielding.” The entire content of U.S. Pat. No. 5,827,997 is hereby incorporated by reference.

In alternative embodiments, the conductive fiber is a metallic fiber comprising a metal or metal alloy. The metallic fiber is coated with a carbon, ceramic or resin material, thereby producing a composite conductive fiber.

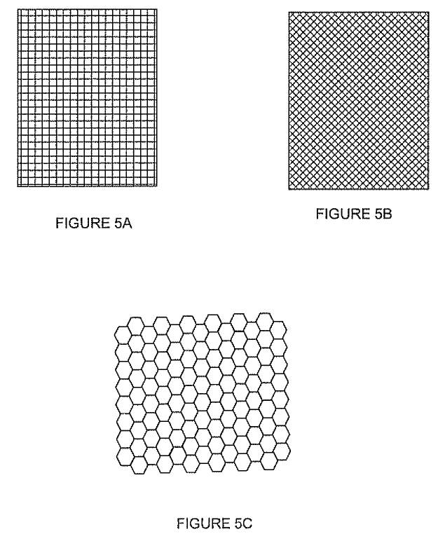

FIGS. 5A-C illustrate various weaves that may be employed in the RF energy absorbing fabric. As shown in FIG. 5A, in one embodiment, the fabric comprises a plurality of fibers with substantially perpendicular weave. In general, however, the mesh may comprise any type of suitable pattern, stitch or weave known to one of skill in the art. For example, in further embodiments, the plurality of fibers form a diagonal cross-type weave (FIG. 5B) or a tricot type weave (FIG. 5C). As defined herein, a tricot type weave is any weave that incorporates the knitting of three threads. Typically, a tricot type weave forms hexagonal apertures in a fabric.

In one embodiment, the fabric is laminated to a first layer of material. In general, this first layer is a thermally insulating layer used to provide comfort for the patient and to insulate the patient from any heat generated by RF absorption. Thus, the first layer is preferably disposed between the patient's skin and the RF absorbing fabric. The first layer may be made from any suitable polymeric material. Examples of suitable materials include without limitation, nylon, Gore-Tex, polyester, polypropylene, polyethylene, polyurethane, polyvinylchloride, or combinations thereof. In another embodiment, first layer comprises a natural fabric such as cotton, silk, wool, or combinations thereof.

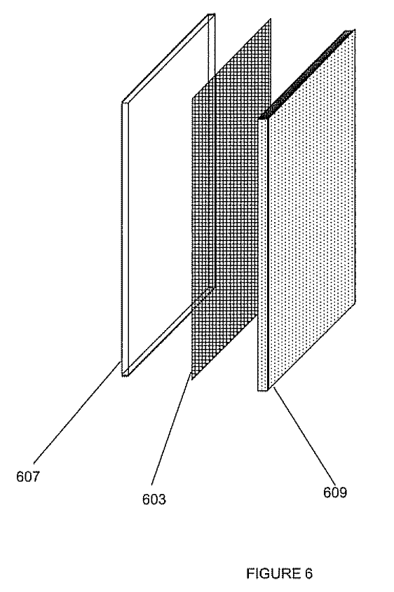

In another embodiment, shown in FIG. 6, an RF energy absorbing fabric 603 is disposed between a first layer 607 as described above, and second layer 609 so as to form a laminate. First and second layers 607, 609 may be constructed from the same or different materials. As is the case for first layer, second layer 609 may be constructed from a polymeric material or a natural fabric. In a particular embodiment, the second layer 609 is ripstop nylon to prevent fraying or tearing of the RF absorbing fabric. Generally, first and second layer 607, 609 may be laminated to the carbon fiber fabric by any method known to one of skill in the art. In a further embodiment, the RF shield comprises more than one carbon fabric layer (not shown). Each carbon fiber fabric layer is preferably disposed between non-carbon fiber layers. The additional carbon fiber fabric layers further enhance the absorption of RF energy.

Although the present invention and its advantages have been described in detail, it should be understood that various changes, substitutions and alterations may be made herein without departing from the spirit and scope of the invention as defined by the appended claims.

What is claimed is:

1. An RF energy absorbing shield comprising:
a fabric comprising a plurality of carbon fibers, wherein said fabric circumferentially surrounds a portion of a patient's body so as to reduce heating of an implantable medical device inside said portion of said patient's body due to RF energy.

2. The RF energy absorbing shield of claim 1, wherein said carbon fiber fabric forms a garment.

3. The RF energy absorbing shield of claim 2, wherein said garment is sleeveless.

4. The RF energy absorbing shield of claim 2, wherein said garment substantially covers the upper torso and the neck of said patient.

5. The RF energy absorbing shield of claim 2, wherein said garment substantially covers at least a portion of a single appendage of said patient.

6. The RF energy absorbing shield of claim 1, wherein said implantable medical device is a vagus nerve stimulator.

7. The RF energy absorbing shield of claim 1, wherein said fabric is capable of reducing RF energy reaching the implantable medical device by at least 50%.

8. The RF energy absorbing shield of claim 1, wherein said fabric is capable of absorbing RF energy in the range of about 40 MHz to about 20 GHz.

9. The RF energy absorbing shield of claim 1, wherein the plurality of carbon fibers are spaced between about 0.001 mm to about 2.0 cm apart.

10. The RF energy absorbing shield of claim 1, wherein said fabric comprises a mesh.

11. The RF energy absorbing shield of claim 1, wherein said fabric comprises a weave selected from the group consisting of a diagonal cross-weave or a tricot weave.

12. The RF energy absorbing shield of claim 1, wherein said fabric further comprises a plurality of conductive metal fibers.

13. The RF energy absorbing shield of claim 1, further comprising
a first layer, wherein said fabric is laminated to said first layer and said first layer is disposed between said fabric and the skin of said patient.

14. The RF energy absorbing shield of claim 13, further comprising
a second layer, wherein said fabric is disposed between said first layer and said second layer.

15. The RF energy absorbing shield of claim 13, wherein said first layer is thermally insulating.

16. The RF energy absorbing shield of claim 14, wherein said first layer and said second layer comprises a material selected from the group consisting of cotton, wool, silk, nylon, polyester, polypropylene, polyethylene, polyurethane, polyvinylchloride, polytetrafluoroethylene, or combinations thereof.

17. The RF energy absorbing shield of claim 14, wherein said second layer is ripstop nylon.

18. An RF energy absorbing shield comprising:
a fabric comprising a plurality of conductive metal fibers, wherein said fabric circumferentially surrounds a portion of a patient's body so as to reduce heating of an implantable medical device inside said portion of a patient's body due to RF energy.

19. A method, comprising:
providing an RF shield made of an RF absorbing fabric; and
circumferentially surrounding a portion of a patient with said RF shield so as to reduce heating of an implantable medical device inside said patient due to RF energy.

20. The method of claim 19, wherein circumferentially surrounding a portion of a patient comprises surrounding the neck and upper torso of the patient with the RF shield.
